(19) United States
(12) Patent Application Publication
(10) Pub. No.: US 2018/0344515 A1
(43) Pub. Date: Dec. 6, 2018

(54) THERMOREGULATORY GLOVE AND METHOD FOR PRODUCING A CONVERGENCE IN BODY TEMPERATURE

(71) Applicants: Parker-Hannifin Corporation, Cleveland, OH (US); Cornell University, Ithaca, NY (US)

(72) Inventors: Matthew Ebben, Forest Hills, NY (US); Michael Bunyan, Newburyport, MA (US); Gino Banco, Chardon, OH (US); William Lionetta, Harwichport, MA (US); George Spies, Wakefield, MA (US); Marc Larosee, Wilmington, MA (US); Steven Bythrow, Weymouth, MA (US); Andrew Douglas Sylvester, Richmond, VA (US)

(21) Appl. No.: 15/997,186

(22) Filed: Jun. 4, 2018

Related U.S. Application Data
(63) Continuation of application No. 62/515,040, filed on Jun. 5, 2017.

(51) Int. Cl. A61F 7/02; A61F 7/00

(52) U.S. Cl. CPC A61F 7/02; A41D 19/00; A61F 7/007

(57) ABSTRACT

The present disclosure provides for a thermoregulatory glove for reducing sleep latency. The thermoregulatory glove includes a gel pocket with a gel pack disposed inside of the gel pocket. A method for producing a convergence in body temperature of a subject includes heating the thermoregulatory glove, placing the heated thermoregulatory glove on a hand of the subject, and cooling the thermoregulatory glove.

This application claims the benefit of U.S. Provisional Application No. 62/515,040 filed Jun. 5, 2017, which is hereby incorporated by reference in its entirety.

### TECHNICAL FIELD

The present disclosure relates generally to an apparatus and method for reducing sleep latency, and more particularly to a thermoregulatory glove and a method for producing a convergence in body temperature.

### BACKGROUND

Sleep disruption and irregularities, including insomnia, are a widespread problem. Various devices and medications have been developed to treat insomnia and reduce sleep latency.

### SUMMARY

The present disclosure provides for a thermoregulatory glove for reducing sleep latency. The thermoregulatory glove includes a gel pocket with a gel pack disposed inside of the gel pocket. A method for producing a convergence in body temperature of a subject includes heating the gel pack of the thermoregulatory glove, placing the heated gel pack contained within the thermoregulatory glove on a hand of the subject, and cooling the gel pack of the thermoregulatory glove.

In accordance with one aspect of the present disclosure, a thermoregulatory glove includes: a glove body having a front hand portion, a back hand portion, a chamber defined by the front hand portion and the back hand portion, and an opening for providing access to the chamber; a gel pocket disposed along the back hand portion of the glove body; and a gel pack disposed inside of the gel pocket.

In one embodiment, the glove body is made of a polyester mesh.

In another embodiment, the thermoregulatory glove further includes support straps.

In another embodiment, the gel pack includes a single layer bladder and a thermally conductive gel disposed inside of the bladder.

In another embodiment, the single layer bladder comprises polypropylene.

In another embodiment, the chamber comprises finger regions and a thumb region.

In another embodiment, at least one of the finger and thumb pockets is open distally.

In accordance with another aspect of the present disclosure, a method for producing a convergence in body temperature of a subject using a thermoregulatory glove having a gel pack includes: (a) heating the gel pack of the thermoregulatory glove to a temperature that is higher than the body temperature of the subject; (b) placing the gel pack contained within thermoregulatory glove on a hand of the subject, wherein the contact between the heated gel pack of the thermoregulatory glove and the hand of the subject increases the temperature of the hand; (c) cooling the gel pack of the thermoregulatory glove, wherein the contact between the cooled gel pack and the hand of the subject decreases the temperature of the hand.

In one embodiment, step (a) occurs before step (b).

In another embodiment, step (a) and step (b) occur simultaneously.

In another embodiment, in step (a), the thermoregulatory glove is heated to a temperature of about 37° C.

In another embodiment, in step (a), the heating is performed with a clam shell heater.

In another embodiment, in step (b), the thermoregulatory glove is placed on the hand such that the gel pack contacts the back of the subject's hand.

### BRIEF DESCRIPTION OF THE DRAWINGS

FIG. 1 is an oblique view of an exemplary thermoregulatory glove according to the disclosure with the gel pack removed.

FIG. 2 is an oblique view of the thermoregulatory glove of FIG. 1 with a wearer's hand inserted therein.

FIG. 3 is an oblique, palm-side up view of the thermoregulatory glove of FIG. 2 as secured to a wearer's hand.

FIG. 4 is an oblique, palm-side down view of thermoregulatory glove of FIG. 2 as secured to a wearer's hand.

FIG. 5 is an oblique view of another embodiment of a thermoregulatory glove according to the disclosure with the gel pack removed.

FIG. 6 is an oblique, palm-side down view of the thermoregulatory glove of FIG. 5 with a wearer's hand therein.

FIG. 7 is an oblique, palm-side up view of thermoregulatory glove of FIG. 5 with a wearer's hand therein.

FIG. 8 is a block diagram of another embodiment of the thermoregulatory glove according to the present disclosure, wherein a biofeedback system is incorporated into the glove.

FIG. 9 is a flow chart showing an exemplary method of producing convergence in body temperature according to the disclosure.

FIG. 10 is an oblique view of an open clam shell heater used to heat the thermoregulatory glove of the present disclosure.

FIG. 11 is an oblique view of the clam shell heater of FIG. 10 in a closed position.

FIG. 12 is an oblique view of another embodiment of the thermoregulatory glove of the present disclosure, wherein the glove includes an internal programmable heater and/or cooler.

FIG. 13 is an oblique view of another embodiment of the thermoregulatory glove of the present invention, wherein the glove includes a negative pressure cuff.

FIG. 14 is a graph of the temperature profile of various subjects wearing the thermoregulatory glove of FIGS. 1-4, indicated by a thermocouple taped to the back-center of the subject's hand.

### DETAILED DESCRIPTION

In the description that follows, to illustrate embodiments of the present disclosure in a clear and concise manner, the drawings may not necessarily be to scale and certain features may be shown in somewhat schematic form. Features that are described and/or illustrated with respect to one embodiment may be used in the same way or in a similar way in one or more other embodiments and/or in combination with or instead of the features of the other embodiments.

Insomnia affects anywhere from six percent to 33% of the U.S. population depending on the precision of the definition being used. Therefore, insomnia represents one of the most prevalent health concerns for Americans. Individuals with insomnia frequently report problems such as (but not limited to) difficulty focusing and concentrating, memory difficulties, impaired motor coordination, irritability and impaired social interactions. Moreover, chronic insomnia has also been associated with reduced quality of life, higher absenteeism, impaired job performance, and higher healthcare utilization.

Sleep and thermoregulation are both functions that have periodic fluctuations in tune with the light-dark cycle and are considered to be regulated by an endogenous circadian oscillator. It has been hypothesized that normal sleep involves a two-process regulation. Sleep onset occurs because of both an increase in homeostatic drive that builds up throughout the waking period as well as the diminution of the circadian arousal process. One of the main circadian indicators is core body temperature, which (under normal entrainment conditions) reaches its zenith in mid-afternoon and slowly declines throughout the evening prior to sleep and precipitously falls after sleep onset.

The association between sleep and thermoregulation is further reinforced by studies that have investigated both passive and active body heating on sleep. Investigators have found that both exercise (which increases core body temperature) and hot baths (if performed in relatively close temporal proximity to sleep) are associated with an increase in slow wave sleep and subjective sleepiness.

An exemplary thermoregulatory glove includes a gel pocket with a gel pack disposed inside of the gel pocket. The thermoregulatory glove is heated and placed on a hand of a subject. Warming of the hand dilates blood vessels, which stimulates blood flow and reduces the temperature difference between the core and the distal limbs of the subject. The thermoregulatory glove is cooled. This results in cooler blood flowing from the hand to the core, which reduces the overall body temperature and results in a convergence of the temperature of the subject's hand and core. Moreover, if evening exercise is controlled to prevent increases in body temperature, increases in slow wave sleep do not occur. Therefore, it appears that increases in body temperature underlie the changes in sleep and not the increase in activity.

The present disclosure is directed to an apparatus and method for reducing sleep latency, and more particularly to a thermoregulatory glove and a method for producing a convergence in body temperature.

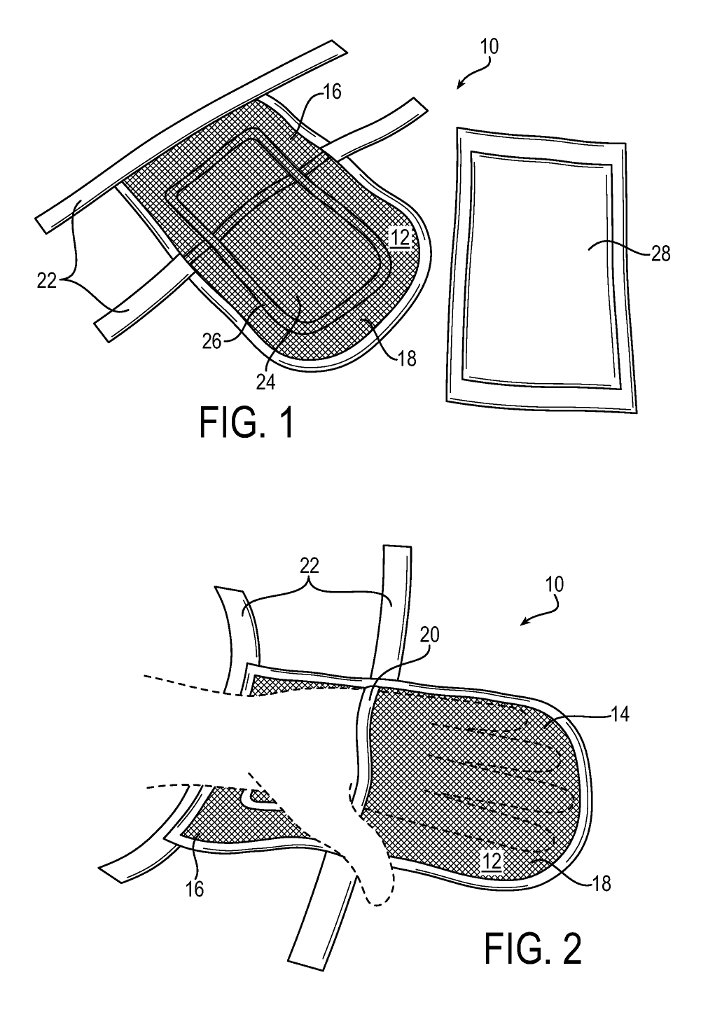

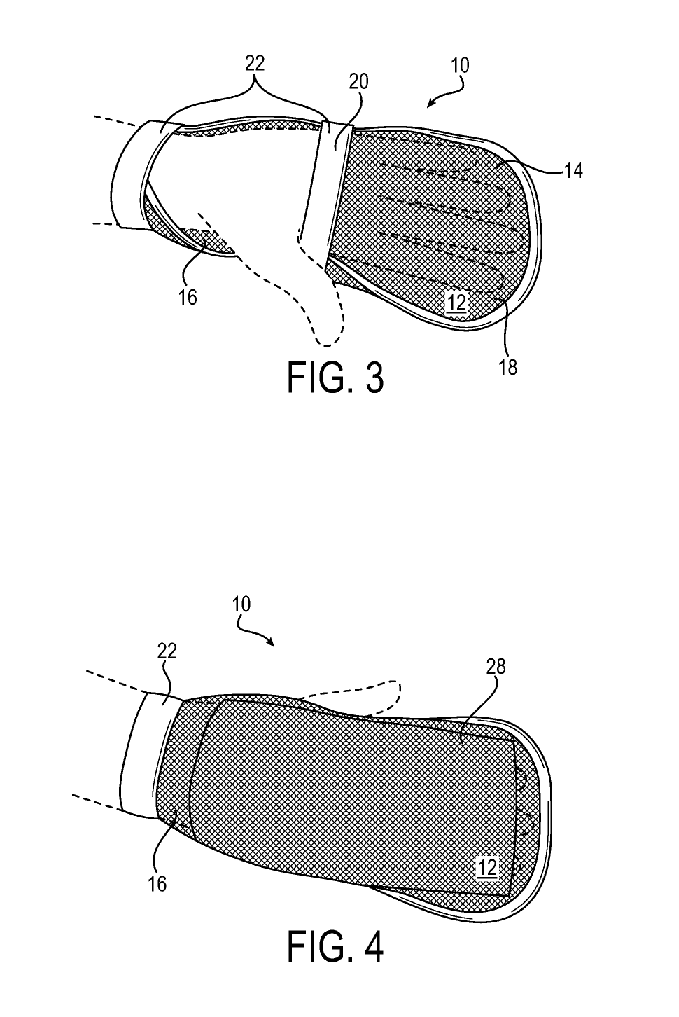

Referring now in detail to the drawings and initially to FIGS. 1-4, an exemplary thermoregulatory glove is shown at 10. The thermoregulatory glove includes a glove body 12 having a front hand portion 14 and a back hand portion 16. The front hand portion 14 and the back hand portion 16 define a chamber 18. The chamber 18 is configured to accept a subject's hand. An opening 20 provides access to the chamber 18.

As shown in FIGS. 1-4, the thermoregulatory glove 10 may be made of a polyester mesh. In some embodiments, the thermoregulatory glove 10 may be made of a hard or semi-rigid plastic that could be custom heat formed or sized to conform to an individual subject's hand. In other embodiments, the thermoregulatory glove may be made of an elastic material (e.g., spandex), which would stretch and conform to a variety of individual subject's hand sizes and shapes. In other embodiments, the thermoregulatory glove 10 may be made of antimicrobial material to minimize the long term susceptibility to microbial growth.

Support straps 22 secure the glove body 12 to a hand and/or a portion of a lower arm. As depicted in FIGS. 1-4, the straps 22 may be made of Velcro®. In other embodiments, the straps 22 may be made of snaps, magnetic fasteners, elastic materials, or a combination of two or more thereof.

A gel pocket 24 is disposed along the back hand portion 16 of the glove body 12. The gel pocket 24 overlies a metacarpal portion of the back of the hand where most of the vasculature of the hand is located. The gel pocket 24 may be formed separately from, or integral to, the back hand portion 16 of the glove body 12. The gel pocket may have a closed circumferential margin 26 defined by annealing, e.g., by stitching or gluing.

A gel pack 28 is disposed within the gel pocket 24 adapted to removably receive the gel pack 28. The gel pack 28 may be in any shape so long as an area of the hand is sufficiently contacted to effect a temperature change. For example, the shape of the gel pack 28 may be square, rectangular, triangular, oval, or circular. The gel pack 28 may be made of a single layer bladder constructed of a flexible, durable material that is resistant to heat, cold, and rupture. The bladder may be fabricated from a variety of material having suitable flexibility, strength and durability to provide a supple, flexible feel when the gel pack 28 is placed within the glove 10. In one embodiment, the bladder may be made of polypropylene, which is used to achieve the necessary heating and cooling profile of the disclosure. Polypropylene is conformable to the hand and has a proper density for heat transfer requirements. The higher thermal conductivity polypropylene allows the necessity for less mass of material, which serves an ergonomic advantage.

A thermally conductive gel may be packaged inside the bladder. Gels useful as the polymer gel component of the invention include gels based on silicones, i.e., polysiloxanes, such as polyorganosiloxane, as well as gels based on other polymers, which may be thermoplastic or thermosetting, such as polyurethanes, polyureas, fluoropolymers, chlorosulfonates, polybutadienes, butyls, neoprenes, nitrites, polyisoprenes, and buna-N, copolymers such as ethylene-propylene (EPR), styrene-isoprene-styrene (SIS), styrene-butadiene-styrene (SBS), ethylene-propylene-diene monomer (EPDM), nitrile-butadiene (NBR), styrene-ethylene-butadiene (SEB), and styrene-butadiene (SBR), and blends thereof such as ethylene or propylene-EPDM, EPR, or NBR. Suitable thermal gels include the THERM-A-GAP™ gel products, which are highly conformable, pre-cured, single-component compounds.

As used herein, the terms “polymer gel” or “polymeric gel” generally have their conventional meaning of a fluid-extended polymer system which may include a continuous polymeric phase or network, which may be chemically, e.g., ionically or covalently, or physically cross-linked, and an oil, such as a silicone or other oil, a plasticizer, unreacted monomer, or other fluid extender which swells or otherwise fills the interstices of the network. The cross-linking density of such network and the proportion of the extender can be controlled to tailor the modulus, i.e., softness, and other properties of the gel. The term “polymer gel” or “polymeric gel” should also be understood to encompass materials which alternatively may be classified broadly as pseudogels or gel-like having viscoelastic properties similar to gels, such as by having a “loose” cross-linking network formed by relatively long cross-link chains, but as, for example, lacking a fluid-extender.

In accordance with one aspect of the present invention, the polymer gel component is rendered thermally-conductive by loading the gel with a filler component which may comprise one or more thermally-conductive particulate fillers. In this regard, the polymer gel component generally forms a binder into which the thermally-conductive filler is dispersed. The filler is included in proportion sufficient to provide the thermal conductivity desired for the intended application, and generally will be loaded in an amount of between about 20% and about 80% by total weight of the compound. The size and shape of the filler is not critical for the purposes of the present invention. In this regard, the filler may be of any general shape, referred to broadly as “particulate,” including solid or hollow spherical or microspherical flake, platelet, irregular, or fibrous, such as chopped or milled fibers or whiskers, but preferably will be a powder to assure uniform dispersal and homogeneous mechanical and thermal properties. The particle size or distribution of the filler typically will range from between about 0.01 mil to about 10 mil (0.25 μm-250 μm), which may be a diameter, imputed diameter, length, or other dimension of the particle, but may further vary depending upon the thickness of the gap to be filled. If desired, the filler may be electrically-nonconductive such that the compound may be both dielectric or electrically-insulating and thermally-conductive. Alternatively, the filler may be electrically-conductive in applications where electrical isolation is not required.

Suitable thermally-conductive fillers include metal oxides, nitrides, carbides diborides, as well as graphite, graphene, metal particles and mixtures thereof, and more particularly boron nitride, titanium diboride, barium titanium borate, aluminum nitride, silicon carbide, graphite, metals such as silver, aluminum, and copper, metal oxides such as aluminum oxide, magnesium oxide, zinc oxide, beryllium oxide, and antimony oxide, and mixtures thereof. Such fillers characteristically exhibit a thermal conductivity of at least about 20 W/m-K. For reasons of economy, an aluminum oxide, i.e., alumina, may be used, while for reasons of improved thermal conductivity a boron nitride may be preferred.

In other embodiments, the gel pack 28 may include a molded thermally conductive material, a TPE, thermally conductive plastic, a metal powder, rice, grains, or a layered composite of said materials.

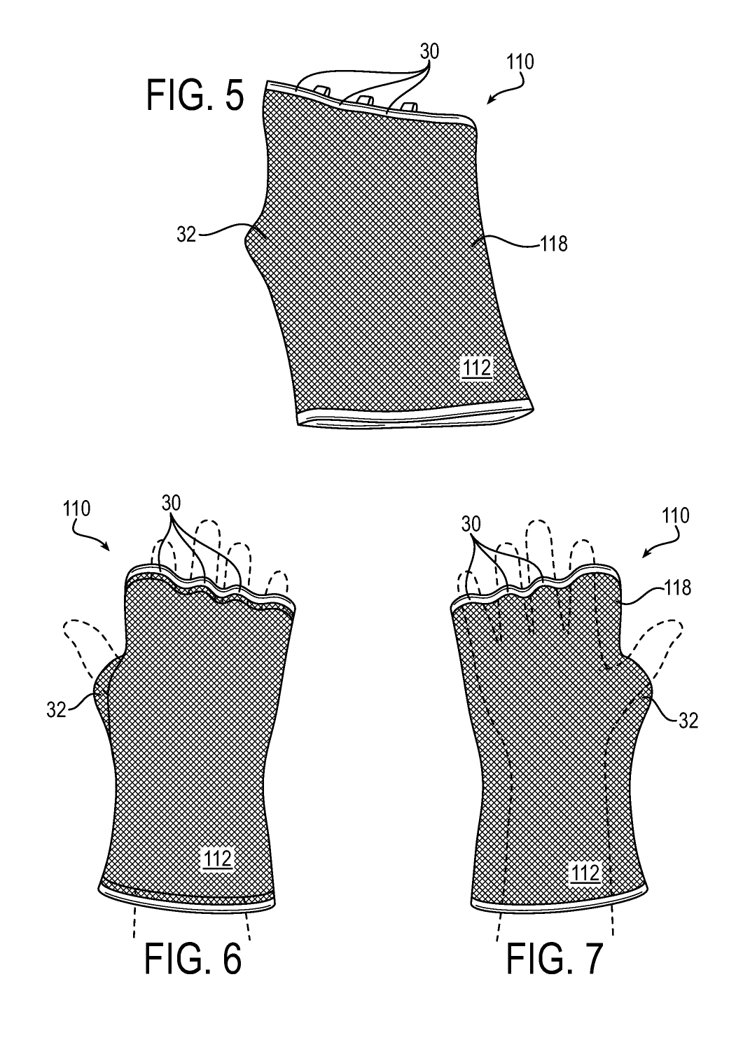

Referring now to FIGS. 5-7, the chamber 118 of the glove body 112 of the thermoregulatory glove 110 includes finger regions 30 and a thumb region 32. As shown in FIGS. 5-7, at least one of the finger and thumb regions 30 and 32, respectively, may be open distally to allow the subject to maintain touch sensation while sleeping.

In some embodiments, the thermoregulatory glove 10 or 110 may include a carrying case for portability, power adapters for the system, LED or sound alters to alert the subject that the system is up to temperature, built in massage or sound such as nature, white noise or other relaxing sounds.

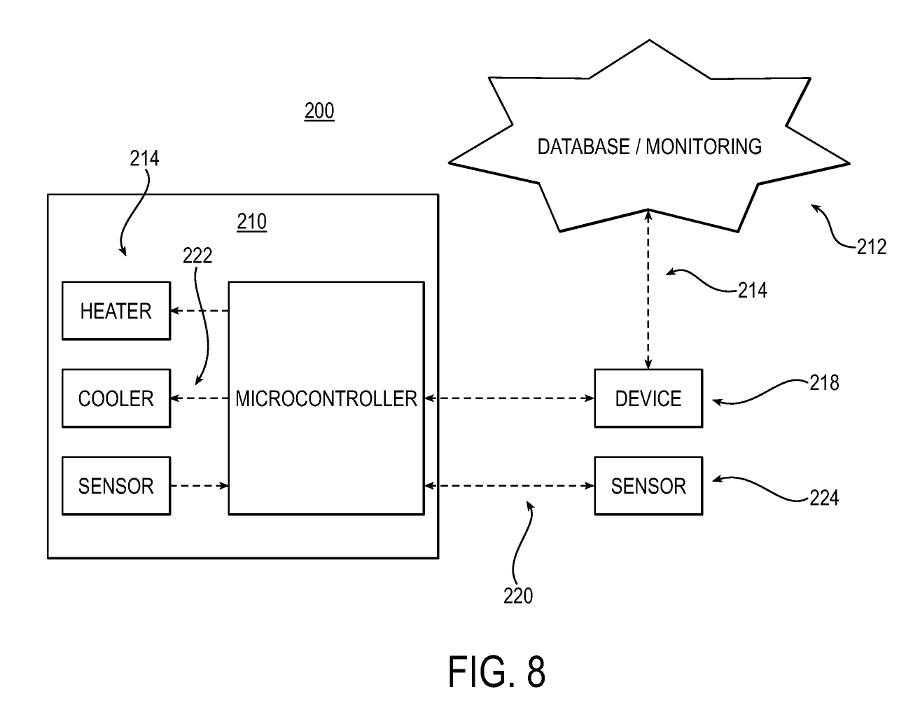

Referring now to FIG. 8, a biofeedback system 200 is incorporated into the thermoregulatory glove 210 to monitor and record sleep rhythms and subject vitals. Such a system 200 includes a storage and monitoring mechanism 212 used to provide feedback 214 to the subject's physician or to automatically control the heating and cooling settings 216 on the system to adapt to the individual subject's needs. In some embodiments, the biofeedback system 200 may interface directly with a hand-held internet-connected device, such as a smartphone based controller 218, or alternatively interface through a separate internet gateway device. The device 218 may include interface 220 with a microcontroller 222 connected to sensors including temperature, pressure, accelerometer, etc., that may be embedded within the device glove 210 for monitoring. The device 210 may also interface with ambient sensors 224 including sound, light, temperature, etc. In another embodiment, a smart fabric may be utilized to provide feedback and sensing capabilities. In other embodiments, the thermoregulatory glove 210 may interface with a hand-held or phone based controller or application that may allow the subject to set an alarm for the following morning, which may then be translated to provide an alternative cycle of operations to allow for optimized waking.

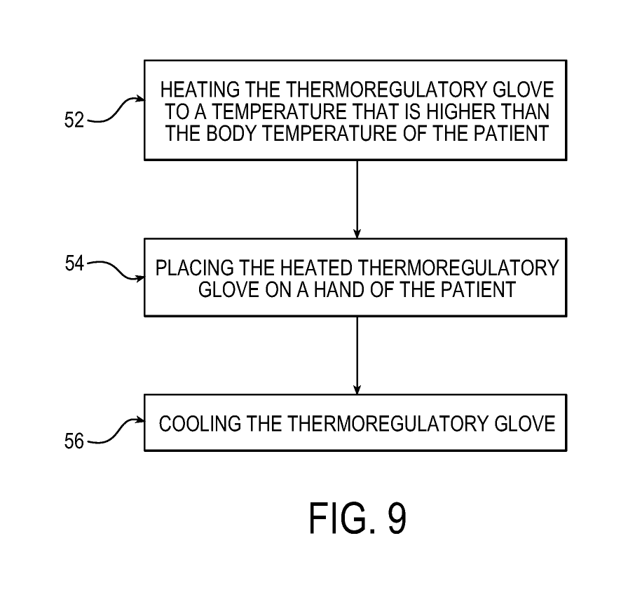

Referring now to FIG. 9, a method for producing a convergence in body temperature is shown at 50. At step 52, the thermoregulatory glove 10 (FIG. 1) is heated to reach a specified temperature, T₁, which is higher than body temperature, T_{B}. The average normal body temperature is 37° C. In a preferred embodiment, T₁is 37° C., while T_{B} is 34° C. In one embodiment, T₁ is in the range of about 25° C. to 45° C., or in another embodiment, in the range of about 30° C. to 40° C., or in another embodiment, in the range of about 34° C. to 38° C., or in another embodiment, in the range of about 35° C. to 40° C., or in another embodiment, in the range of about 36° C. to 38° C. In one embodiment, T_{B} is in the range of about 30° C. to 40° C., or in another embodiment, in the range of about 32° C. to 38° C., or in another embodiment, in the range of about 35° C. to 40° C., or in another embodiment, in the range of 33° C. to 35° C., or in another embodiment, in the range of 36° C. to 38° C.

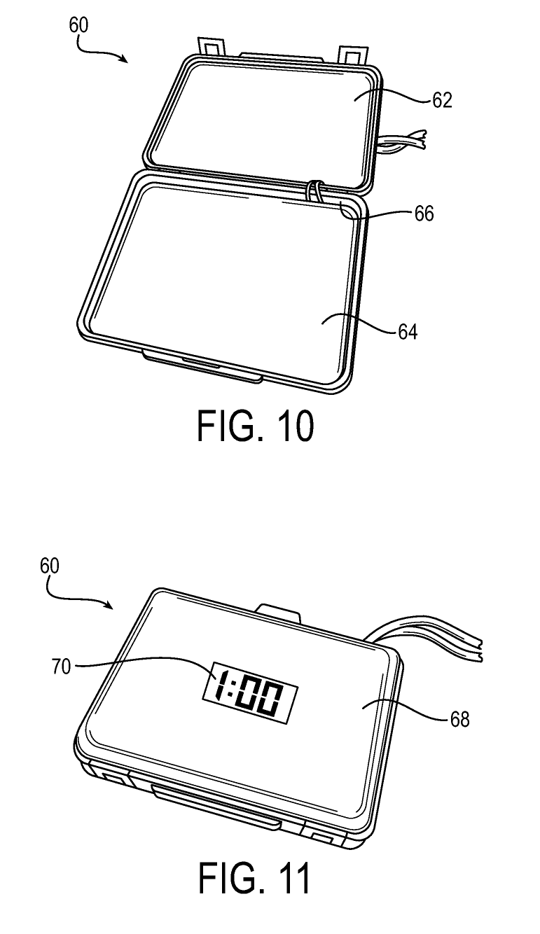

The thermoregulatory glove 10 (FIG. 1) may be heated using a variety of external methods such as conduction, convection, insulation, free convection, radiation, or a combination of two or more thereof to obtain the specified temperature T₁. For example, referring to FIGS. 10 and 11, a clam shell heater is shown at 60. The clam shell heater includes an upper resistive heater 62, a lower resistive heater 64, insulation 66, and an outer case 68. The heater 60 includes a timer 70 for setting the heat time and an alarm notifying the user when the thermoregulatory glove 10 (FIG. 1) is heated to the specified temperature T₁. The timer 70 may be activated when the gloves are placed on the heater 60. In one embodiment, a display or illumination may show if the box is warm/on and when the glove 10 has reached the specified temperature T₁. In another embodiment, the heater 60 could have an active temperature controller that ensures proper heating of the glove 10 to the specified temperature T₁.

In other embodiments, the thermoregulatory glove 10 (FIG. 1) may be heated using boiling water, a microwave, a hot plate, or a combination of two or more thereof. These methods may heat the thermoregulatory glove 10 from a single surface, such as by placement onto a heating pad, or from all sides, such as a claim shell type heater as shown in FIGS. 9 and 10 or submersion in boiling water or other heated fluid. In one embodiment, heat is generated through the utilization of the energy released by an exothermic chemical reaction. In some embodiments, the thermoregulatory glove may be heated with a peltier device that may allow for cooling of the thermoregulatory glove 10.

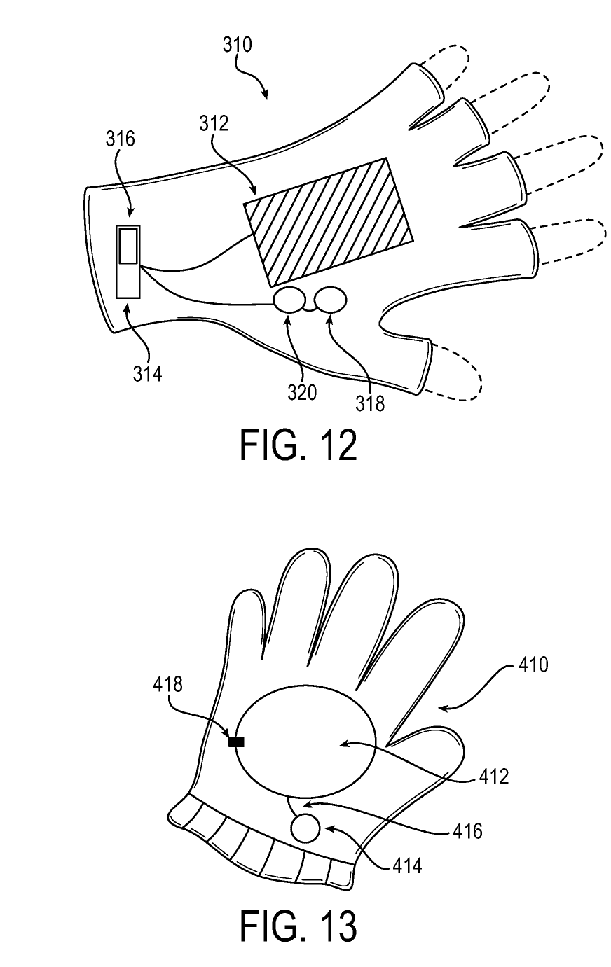

Referring to FIG. 12, the thermoregulatory glove 310 includes an internal programmable heater and/or cooler 312. The internal programmable heater and/or cooler 312 may be made of resistive wires or of thermoelectric heating and/or cooling elements. In some embodiments, the thermoregulatory glove 310 may include internal tubing and a recirculation system coupled with a temperature regulated fluid to heat the thermoregulatory glove 310 to the specified temperature T₁. The heater and/or cooler 312 may interface with microcontroller 314 that may be connected to a heart rate sensor 316. The microcontroller 314 may also interface with a sensor package 318 that could include a temperature sensor, accelerometer, gyroscope, etc. The subject may activate and deactivate the heating and/or cooling component with a switch 320 which may interface with the microcontroller 314. Various methods of cooling may be used such as a gel or a material that is placed in the freezer or a fluid cooling system.

In some embodiments, other forms of vasodilation, such as heating, negative pressure, and/or a pharmaceutical agent, may be used prior to heating and/or cooling in order to alter a subject's core body temperature. For example, a subject may apply a vasodilating cream to his hand for a certain period of time prior to placement of the thermoregulatory glove 10 (FIG. 1) that has a cooling effect on the subject's core body temperature.

Referring to FIG. 13, in another embodiment, a glove 410 includes a negative pressure cuff 412 located within the glove 410. The cuff 412 may be evacuated by any acceptable means such as, but not limited to, a hand pump 414, a miniature electrical pump, or other manual forms of suction such as a pulling back a syringe through an on-board, valved port. Tubing 416 may provide an interface between the cuff 412 and the manual pump 414. The cuff may also include a regulated valve or syringe port 418 to de-pressurize over a chosen time interval.

Referring back to FIG. 9, at step 54, the thermoregulatory glove 10 (FIG. 1) is placed on a hand of the subject. The thermoregulatory glove 10 may be placed on the hand of the subject such that the gel pack 28 is in contact with the back of the subject's hand where most of the vasculature is located. Step 54 may occur before, simultaneous with, or after step 52. The contact between the heated thermoregulatory glove 10 and the hand causes the temperature of the subject's hand to increase. This increase in hand temperature causes the blood vessels to dilate. Dilated blood vessels carry more blood than non-dilated vessels. Thus, the dilated vessels enable the hand to serve as a more effective heat exchanger by stimulating blood flow to and from the hand. This causes the subject's body core and distal temperatures to equilibrate (i.e., warm blood flowing from the hands to the core).

At step 56, with no additional heat added to the thermoregulatory glove 10 (FIG. 1), the thermoregulatory glove 10 begins to cool, causing the hands to cool, which causes the blood to cool. In some embodiments, the hands may be cooled via conductive properties of the material inside the glove or by a cooling mechanism. Because the blood vessels are still dilated, cool blood flows from the subject's hand to the body core in an accelerated period of time. This reduces the subject's overall body temperature (i.e., causes a convergence between the subject's body core and distal temperatures), which induces sleep.

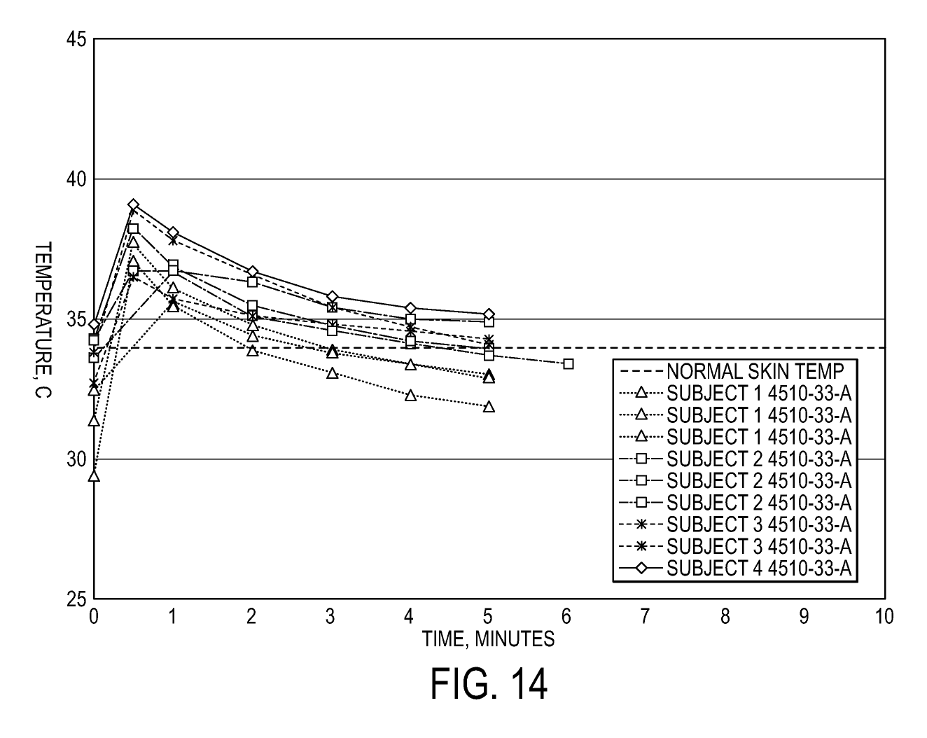

Referring now to FIG. 14, one embodiment of the glove can track the temperature profile of individual subjects through the attachment of a thermocouple to the back-center of a subject's hand. As depicted in the graph, the subjects experience skin temperature increases followed by temperature decreases, as previously described in FIG. 9.

The present disclosure provides for a thermoregulatory glove 10 for reducing sleep latency. The thermoregulatory glove includes a gel pocket 24 with a gel pack 28 disposed inside of the gel pocket 24. A method for producing a convergence in body temperature of a subject includes heating the thermoregulatory glove 10, placing the heated thermoregulatory glove on a hand of the subject 10, and cooling the thermoregulatory glove 10.

Although the invention has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A method for producing a convergence in body temperature of a subject, the method comprising:
(a) providing a thermoregulatory glove comprising:
a glove body having a front hand portion, a back hand portion, a chamber defined by the front hand portion and the back hand portion, and an opening for providing access to the chamber;
a gel pack; and
a pocket disposed along the back hand portion of the glove body for containing the gel pack; and
(b) heating the gel pack of thermoregulatory glove to a temperature that is higher than the body temperature of the subject;
(c) placing the thermoregulatory glove with the gel pack contained within the pocket on a hand of the subject, wherein the contact between the heated gel pack of thermoregulatory glove and the hand of the subject increases the temperature of the hand;
(d) cooling the gel pack of the thermoregulatory glove, wherein the contact between the cooled gel pack of the thermoregulatory glove and the hand of the subject decreases the temperature of the hand.

2. The method of claim 1, wherein step (b) occurs before step (c).

3. The method of claim 1, wherein step (b) and step (c) occur simultaneously.

4. The method of claim 1, wherein step (b) occurs after step (c).

5. The method according to claim 1, wherein in step (b), the gel pack of the thermoregulatory glove is heated to a temperature in the range of about 35° C. to 40° C.

6. The method according to claim 1, wherein in step (b), the heating is performed with a clam shell heater.

7. The method according to claim 1, wherein in step (c), the thermoregulatory glove is placed on the hand such that the gel pack contacts the back of the subject's hand.

8. The method of claim 1, wherein the glove body is made of a polyester mesh.

9. The method of claim 1, wherein the thermoregulatory glove further comprises support straps.

10. The method of claim 1, wherein the gel pack comprises a single layer bladder and a thermally conductive gel disposed inside of the bladder.

11. The method of claim 10, wherein the single layer bladder comprises polypropylene.

12. The method of claim 1, wherein the chamber of the glove comprises finger regions and a thumb region.

13. The method of claim 12, wherein at least one of the finger and thumb pockets is open distally.

14. A method for reducing sleep onset latency in a subject, the method comprising:
(a) providing a thermoregulatory glove comprising:
a glove body having a front hand portion, a back hand portion, a chamber defined by the front hand portion and the back hand portion, and an opening for providing access to the chamber;
a gel pack; and
a pocket disposed along the back hand portion of the glove body for containing the gel pack; and
(b) heating the gel pack of thermoregulatory glove to a temperature that is higher than the body temperature of the subject;
(c) placing the thermoregulatory glove with the gel pack contained within the pocket on a hand of the subject, wherein the contact between the heated gel pack of thermoregulatory glove and the hand of the subject increases the temperature of the hand;
(d) cooling the gel pack of the thermoregulatory glove, wherein the contact between the cooled gel pack of the thermoregulatory glove and the hand of the subject decreases the temperature of the hand.
